(19) United States
(12) Patent Application Publication
(10) Pub. No.: US 2014/0178055 A1
(43) Pub. Date: Jun. 26, 2014

(54) INCENSE BURNER

(71) Applicant: HON HAI PRECISION INDUSTRY CO., LTD., New Taipei (TW)

(72) Inventor: PO-CHOU CHEN, Tu-Cheng (TW)

(73) Assignee: HON HAI PRECISION INDUSTRY CO., LTD., New Taipei (TW)

(21) Appl. No.: 13/897,455

(22) Filed: May 20, 2013

(30) Foreign Application Priority Data
Dec. 26, 2012 (TW) 101150044

(51) Int. Cl. A61L 9/03

(52) U.S. Cl. CPC A61L 9/03
USPC 392/390

(57) ABSTRACT

An incense burner includes a burner body, a cover, and at least one light source. The cover is located in the burner body and defines at least one through hole for movably receiving at least one electronic incense stick. The at least one light source is located in the burner body. The at least one light source emits light into the at least one electronic incense stick.

### BACKGROUND

1. Technical Field

The present disclosure relates to incense burners and, particularly, to incense burners being used commonly for both electronic incense sticks and traditional incense sticks.

2. Description of Related Art

Electronic incense stick is a tube having an opaque coating on the outer surface and a transparent bubble on the top end and guide light emitting from a light source to the transparent bubble to simulate a burning traditional incense stick. The light source is located in an electronic burner.

However, the electronic incense sticks cannot be removed from the electronic burners. Therefore, traditional incense sticks that must be burnt are not suitable for electronic incense burners.

Therefore, it is desirable to provide an incense burner, which can overcome the above-mentioned shortcomings

### BRIEF DESCRIPTION OF THE DRAWINGS

FIG. 1 is a schematic view of an incense burner, in accordance with one embodiment.

FIG. 2 is a sectional view of the incense burner shown in FIG. 1.

FIG. 3 is a sectional view of an incense burner, in accordance with another embodiment.

### DETAILED DESCRIPTION

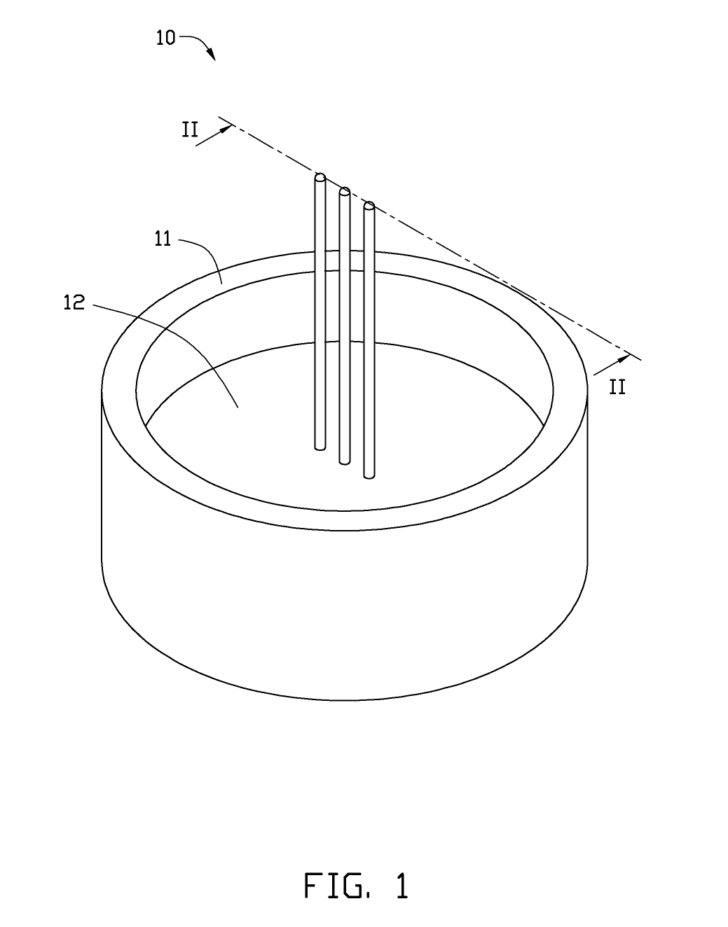

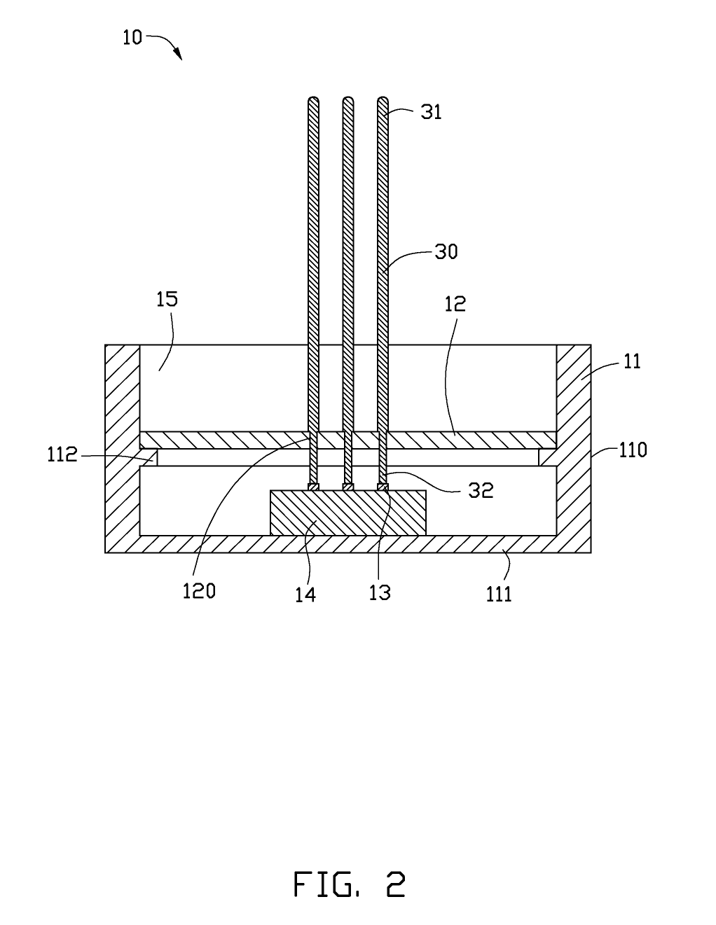

FIGS. 1 to 2 show an incense burner 10 in accordance with one embodiment. The incense burner 10 includes a burner body 11, a cover 12, light sources 13, and a circuit board 14. In this embodiment, electronic incense sticks 30 may be inserted in the incense burner 10. Each of the electronic incense sticks 30 includes a first portion 31 and a second portion 32 and the diameter of the second portion 32 is less than that of the first portion 31. The length of the second portion 32 is less than the length of the first portion 31. Alternatively, traditional incense sticks may also be inserted in the incense burner 10.

The body 11 can be cylinder, or rectangular shapes, for example. The material of the burner body 11 is selected from the group of materials that include stone, copper, and iron, for example. The cover 12 can be also made of stone, copper, or iron, for example.

The burner body 11 includes a side wall 110 and a bottom base 111 connected with the side wall 110. A supporting ring 112 is defined on an inner surface of the side wall 110, thus, the burner body 11 is divided into a top portion and a bottom portion by the supporting ring 112.

The cover 12 is a flat plate and is located on the supporting ring 112. A holding chamber 15 is defined by the cover 12 and the burner body 11. The holding chamber 15 is configured for holding ash generated by the burning traditional incense sticks. Three through holes 120 are defined on the cover 12. The electronic incense sticks 30 are moveably inserted into the through holes 120. One electronic incense stick 30 is inserted into each of through holes 120 or more than one traditional incense stick may be inserted into the same through hole 120. When an electronic incense stick 30 is being used, the electronic incense sticks 30 are inserted into the through holes 120. When using the traditional incense stick, the electronic incense sticks 30 are extracted from the through holes 120 and the traditional incense sticks are inserted into the through holes 120.

The cover 12 is fixed in the burner body 11. The cover 12 is welded or screwed by bolts on the supporting ring 112.

The circuit board 14 is fixed on the bottom base 111 and the light sources 13 are electrically set on the circuit board 14. The number of the light sources 13 is identical to the number of the through holes 120. Each of the light sources 13 corresponds to a through hole 120. The light sources 13 can be light emitting diodes or bulbs.

In practice, the number of the through hole 120 can be one, two, four, etc. The incense burner 10 includes power line connected with the light source 13.

The electronic incense sticks 30 are inserted into the through holes 120, a bottom of the second portion 32 contacts with the light source 13 and the first portions 31 are stopped by the cover 12. Light emitted from the light source 13 couples into and emits out of each of the electronic incense sticks 30 to simulate a traditional burning incense stick.

When the electronic incense sticks 30 are not being used, the light source 13 is powered off and the electronic incense sticks 30 are extracted from the through holes 120, traditional incense sticks are inserted into the through holes 120. Ash generated by the burning of the traditional incense sticks is held in the holding chamber 15.

The cover 12 of the incense burner 10 includes the through holes 120 for movably holding the electronic incense sticks 30 or the traditional incense sticks and the light sources 13 are located in the burner body 11, thus the incense burner 10 can be suitable for both the electronic incense sticks 30 and the traditional incense sticks.

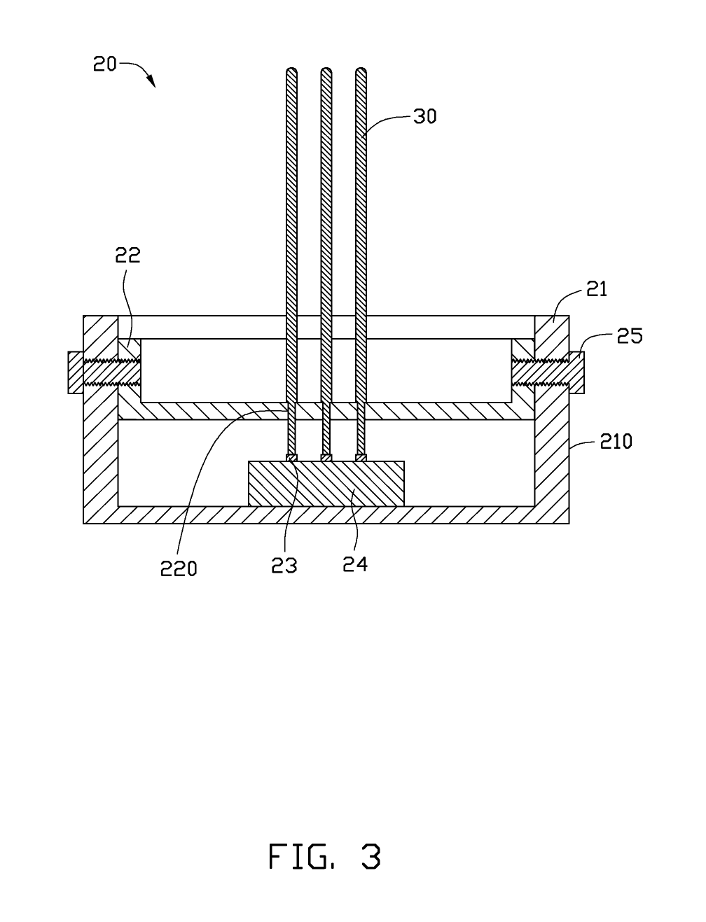

FIG. 3 shows an incense burner 20 in accordance with another embodiment. The incense burner 20 also includes a burner body 21, a cover 22, light sources 23, and a circuit board 24. The burner body 21 includes a side wall 210. The circuit board 24 is located in the burner body 21, and the light sources 23 are set on the circuit board 24. A plurality of through holes 220 are defined in the cover 22. The electronic incense sticks 30 can be movably inserted into the through holes 220. The cover 22 is fixed on the inner surface of the sidewall 210 by screws 25.

In other embodiments, traditional burning incense sticks can also be inserted in the incense burner 20.

Particular embodiments are shown here and described by way of illustration only. The principles and the features of the present disclosure may be employed in various and numerous embodiments thereof without departing from the scope of the disclosure as claimed. The above-described embodiments illustrate the scope of the disclosure but do not restrict the scope of the disclosure.

What is claimed is:

1. An incense burner, comprising:
a burner body;
a cover located in the burner body and comprising at least one through hole for movably receiving at least one electronic incense stick; and
at least one light source located in the burner body, the at least one light source for emitting light into the at least one electronic incense stick.

2. The incense burner of claim 1, wherein the burner body comprises a sidewall and a supporting ring, the supporting ring is positioned on an inner surface of the sidewall, and the cover is located on the supporting ring.

3. The incense burner of claim 2, wherein the cover is movably located on the supporting ring.

4. The incense burner of claim 1, wherein each of the at least one light source is a light emitting diode.

5. The incense burner of claim 1, comprising a circuit board located in the burner body, and the at least one light source being set on the circuit board.

6. The incense burner of claim 1, wherein the cover is fixed on the burner body by screws.

7. The incense burner of claim 1, wherein the material of the burner body is selected from one of the group consisting of copper, iron, and stone.

8. The incense burner of claim 1, wherein the material of the cover is selected from one of the group consisting of copper, iron, and stone.

9. The incense burner of claim 1, wherein each of the at least one electronic incense stick comprises a first portion and a second portion, the second portion is held in a respective one of the at least one through hole, and a diameter of the second portion is less than a diameter of the first portion.
